(19) United States
(12) Patent Application Publication
(10) Pub. No.: US 2011/0104274 A1
(43) Pub. Date: May 5, 2011

(54) NOVEL COMPOSITION AND USES THEREOF, IN PARTICULAR COSMETIC USES, FOR TREATING SKIN DEHYDRATION

(75) Inventors: Quang-Khai Tran, Toussus le Noble (FR); Pascale Rondeau, Savigny Sur Orge (FR); Thierry Saint Nenis, Vincennes (FR); Sandrine Samson-Villeger, Lyon (FR)

(73) Assignee: COMPAGNIE GERVAIS DANONE, Paris (FR)

(21) Appl. No.: 12/445,245

(22) PCT Filed: Oct. 12, 2007
(86) PCT No.: PCT/FR2007/001677

§ 371 Date: Jun. 30, 2009

(30) Foreign Application Priority Data
Oct. 13, 2006 (FR) 0609039

(51) Int. Cl. A61K 31/201; A61K 31/353; A61K 9/28; A61P 17/06; A61P 17/16; A61P 17/00

(52) U.S. Cl. 424/474; 514/560; 514/456

(57) ABSTRACT

The present invention relates to the use of a combination comprising gamma-linolenic acid and at least one green tea polyphenol, in which the ratio of the gamma-linolenic acid to the green tea polyphenol ranges from approximately 1 to approximately 10, for increasing the in vitro and ex vivo differentiation of keratinocytes.

The invention relates to a novel composition and uses thereof, in particular cosmetic uses, for improving the quality of the skin by acting on the “barrier” function of the skin.

The compositions of the invention can be used in particular for treating dehydration of the skin.

The main functions of the skin are in particular to maintain mechanical and chemical protection against radiation and pathogens in the environment, and to prevent loss of water. This “barrier function” of the skin is ensured by the topmost layer of the epidermis: the stratum corneum (SC), corresponding to the final stage of maturation of the keratinocytes. Thus, stimulation of keratinocyte differentiation improves the physiology of the skin and in particular hydration by limiting the losses of water from the skin.

Numerous substances, and in particular gamma-linolenic acid (GLA) and the green tea polyphenols (GTP), are known to have beneficial effects on the skin.

The effects of gamma-linolenic acid (GLA) on the skin can be viewed from 3 aspects. First, GLAs can be incorporated in the cell membranes and the extracellular matrix of the stratum corneum (SC) composed of substances such as fatty acids and ceramides and can thus act on the control of hydration of the skin. Moreover, GLA participates in the regulation of the balance of the eicosanoids of series 1, which have an anti-inflammatory and anti-allergic role. Finally, by activation of the PPAR receptor (peroxisome proliferator-activated receptor) which promotes expression of various maturation proteins and inhibition of DNA synthesis (and therefore of proliferation), GLA might stimulate the maturation of the basal keratinocyte to corneocyte.

The green tea polyphenols (GTPs) improve the “barrier” function of the skin through stimulation of the maturation of the basal keratinocyte to corneocyte. Moreover, GTP and particularly epigallocatechin gallate (EGCG) provides protection against oxidative stress and against free radicals, and stabilization of the membrane of the lysosome, reducing the escape of mediators or of pro-inflammatory enzymes.

The purpose of the present invention is to provide novel compositions having a synergistic effect on the physiology of the skin and in particular on keratinocyte differentiation.

The present invention relates to the use of a combination comprising gamma-linolenic acid and at least one green tea polyphenol, in which the ratio of gamma-linolenic acid to green tea polyphenol varies from approximately 1 to approximately 10, and in particular varies from approximately 2 to approximately 8 and still more particularly from approximately 4 to approximately 6, increasing the in vitro and ex vivo differentiation of keratinocytes.

It was found, unexpectedly, that the combination of gamma-linolenic acid and green tea polyphenols acts beneficially and synergistically on the physiology of the skin, and in particular with respect to keratinocyte differentiation.

A synergistic effect is defined as the effect of a combination of two ingredients which exceeds the addition of the effects that each of these ingredients would have had individually.

By “gamma-linolenic acid” is meant the gamma isomer of linolenic acid, which belongs to the omega 6 fatty acid family. These omega 6 fatty acids are termed “essential” as they are indispensable to our bodies and cannot be synthesized by the body, so must be supplied in our food. Gamma-linolenic acid can be produced from linoleic acid by a series of enzymatic reactions caused by desaturases and elongases and is an essential intermediate in the metabolism of the polyunsaturated fatty acids. Δ6-Desaturase, which is an enzyme that is indispensable to the production of GLA, is an enzyme that does not occur in the cells of the skin (in particular keratinocytes and corneocytes).

By “green tea polyphenols” is meant catechins, the most important of which in green tea is epigallocatechin gallate (40%), followed by epigallocatechin (18%) and then epicatechin (8%). Other minor catechins are present in tea extracts such as gallocatechin (GC), epicatechin gallate (ECG), gallocatechin gallate (GCg) and catechin (C).

The expression “for increasing the in vitro and ex vivo differentiation of keratinocytes” signifies that the composition makes it possible to promote the progression of the keratinocyte in its process of maturation, so that it can best perform its specific functions.

The expression “differentiation of keratinocytes” is to be distinguished from “proliferation”, which corresponds to renewal of the stem cells of the basal layer. The basal cells ensure, by their asymmetric mitoses, both their renewal (maintenance of a contingent of undifferentiated stem cells) and the formation of other basal cells intended for the differentiation process.

According to an advantageous embodiment, the present invention relates to the use, as defined above, of a combination in which the green tea polyphenol is selected from compounds of the flavan-3-ol class of flavonoids.

According to an advantageous embodiment, the present invention relates to the use, as defined above, of a combination in which the green tea polyphenol is selected from epigallocatechin gallate (EGCg), epigallocatechin (EGC), epicatechin (EC), gallocatechin (GC), gallocatechin gallate (GCg), epicatechin gallate (ECg) and catechin (C).

According to an advantageous embodiment, the present invention relates to the use, as defined above, of a combination in which the green tea polyphenol is contained in a plant extract such as green tea extract (GTE).

Green tea extract is obtained according to methods that are fully known to a person skilled in the art (see, among others, standard NF ISO 6079 of September 1991).

According to another advantageous embodiment, the present invention relates to the use, as defined above, of a combination in which the gamma-linolenic acid is contained in a vegetable oil such as borage oil, primrose oil or blackcurrant seed oil.

The GLAs provided by borage oil are in a more bioavailable form than the GLAs provided by evening primrose oil. In borage oil, the GLA is concentrated in position sn-2 of the triglycerides, whereas in evening primrose oil the GLA is concentrated in positions sn-1 and sn-3 of the triglycerides. The enzymes are therefore more easily able to cleave the GLAs of borage oil, which are in a more bioavailable form.

The present invention also relates to a combination comprising gamma-linolenic acid and at least one green tea polyphenol, in which the ratio of gamma-linolenic acid to green tea polyphenol varies from approximately 1 to approximately 10, and in particular varies from approximately 2 to approximately 8 and still more particularly from approximately 4 to approximately 6.

According to an advantageous embodiment, the present invention relates to a combination comprising gamma-linolenic acid and at least one green tea polyphenol, in which the ratio of gamma-linolenic acid to green tea polyphenol varies from approximately 1 to approximately 10, and in particular varies from approximately 2 to approximately 8 and still more particularly from approximately 4 to approximately 6, and in which the total quantity of gamma-linolenic acid and of polyphenol comprises approximately 100 to approximately 500 mg, in particular approximately 150 to approximately 400 mg, more particularly approximately 350 mg.

The present invention relates to a composition comprising a combination as defined above, in which:
the concentration of gamma-linolenic acid is approximately 0.1 to approximately 0.5%, more particularly from approximately 0.1 to approximately 0.3%, still more particularly from approximately 0.15 to approximately 0.3 wt. % relative to the total weight of the composition and
the concentration of green tea polyphenol is approximately 0.01 to approximately 0.1%, more particularly from approximately 0.02 to approximately 0.07%, still more particularly from approximately 0.045 wt. % relative to the weight of the composition.

According to an advantageous embodiment, the present invention relates to a composition as defined above, in which the green tea polyphenol is selected from compounds of the flavan-3-ol class of flavonoids.

According to an advantageous embodiment, the present invention relates to a composition as defined above, in which the green tea polyphenol is selected from epigallocatechin gallate (EGCg), epigallocatechin (EGC), epicatechin (EC), gallocatechin (GC), gallocatechin gallate (GCg), epicatechin gallate (ECg) and catechin (C).

According to another advantageous embodiment, the present invention relates to a composition as defined above, in which the green tea polyphenol is contained in a plant extract such as green tea extract.

According to an advantageous embodiment, the present invention relates to a composition as defined above, in which the gamma-linolenic acid is contained in a vegetable oil such as borage oil, primrose oil or blackcurrant seed oil.

According to an advantageous embodiment, the present invention relates to a composition as defined above, in which the concentration of gamma-linolenic acid is approximately 0.15 to approximately 0.3 wt. % relative to the total weight of the composition.

Outside of these ranges, either no effect is obtained (for concentrations below 0.15%), or problems arise connected with the nutritional and organoleptic balance of the product.

According to an advantageous embodiment, the present invention relates to a composition as defined above, in which the concentration of green tea polyphenol is approximately 0.045 wt. % relative to the total weight of the composition.

This value allows an advantageous effect to be obtained.

According to another advantageous embodiment, the present invention relates to a composition as defined above, in which the concentration of gamma-linolenic acid is approximately 0.15 to approximately 0.3 wt. % relative to the total weight of the composition and the concentration of green tea polyphenol is approximately 0.045 wt. % relative to the total weight of the composition.

The present invention also relates to an oral composition comprising a combination as defined above, said composition being in the form of a food product, a food supplement, or a cosmetic composition.

According to an advantageous embodiment, the present invention relates to an oral composition as defined above, characterized in that the food product is selected from the group comprising a fresh dairy product, a yoghurt, a fromage frais, a fermented dairy product, a dessert, a drink, a liquid, a cream, a fruit and/or vegetable puree.

The expression “fermented milk” or “yoghurt” is to be understood in particular as complying with the official standards of the Codex alimentarius (in particular volume 12 thereof and the standard Codex Stan 1-11 (a)-1975) or French decree No. 88-1203 of 31 Dec. 1988.

According to another advantageous embodiment, the present invention relates to an oral composition as defined above, characterized in that the food supplement is selected from the group comprising coated tablets, pills, gelatin capsules, syrup, gel, powder for reconstitution.

According to another advantageous embodiment, the present invention relates to an oral composition as defined above, characterized in that the cosmetic composition is selected from the group comprising creams, emulsions, lotions, paste, gum.

According to an advantageous embodiment, the present invention relates to an oral composition as defined above, in which the concentration of gamma-linolenic acid is approximately 0.15 to approximately 0.3 wt. % relative to the total weight of the composition and the concentration of green tea polyphenol is approximately 0.045 wt. % relative to the total weight of the composition.

The recommended daily doses of this oral composition are 1 to 2 portions, i.e. corresponding to the ranges from 150 to 300 mg of GLA and from 45 to 90 mg of GTP.

The present invention also relates to a cosmetic product comprising a combination as defined above, in combination with a suitable excipient for administration by the topical route.

By “suitable excipient for administration by the topical route” is meant excipients allowing the active ingredient to reach the stratum corneum.

According to an advantageous embodiment, the present invention relates to a cosmetic product as defined above, in which the concentration of gamma-linolenic acid is approximately 0.15 to approximately 0.3 wt. % relative to the total weight of the composition and the concentration of green tea polyphenol is approximately 0.045 wt. % relative to the total weight of the composition.

The present invention relates to a pharmaceutical composition comprising a combination as defined above, in combination with a pharmaceutically acceptable vehicle.

By “pharmaceutically acceptable vehicle” is meant excipients for carrying the active ingredient to its target.

A subject of the present invention is also a method of cosmetic treatment, comprising the absorption, by the oral route, of a cosmetic composition as defined above.

A subject of the present invention is also a method of cosmetic treatment, comprising the topical application of a cosmetic product as defined above.

The present invention relates to the use of a combination as defined above, for the preparation of a medicament intended to improve the quality of the skin by acting on the “barrier” function of the skin.

The present invention relates to the use of a combination as defined above, for the preparation of a medicament intended for the prevention or treatment of pathologies involving a change, in particular a decrease, in keratinocyte differentiation.

The pathologies involving a change, in particular a decrease in keratinocyte differentiation can be, for example, an atopic dermatitis or psoriasis.

### FIGURES

FIG. 1 shows the percentage expression of transglutaminase K (TGK) in keratinocytes previously treated with GLA and GTP alone and in combination. Column A corresponds to treatment of the keratinocytes with 1.25 μg/ml of GLA. Column B corresponds to treatment of the keratinocytes with 3.75 μg/ml of GLA. Column C corresponds to treatment of the keratinocytes with 0.5 μg/ml of GTP. Column D corresponds to treatment of the keratinocytes with 2.5 μg/ml of GLA and 0.5 μg/ml of GTP. Column E corresponds to treatment of the keratinocytes with calcium at a concentration of 1.5 mM, well known as stimulating keratinocyte differentiation.

FIG. 2 shows the percentage expression of involucrin in the keratinocytes (NHEKs) of women subjects and obtained from the abdominal epithelium (Group 1) or from the epithelium of the breast (Group 2), previously treated with a small quantity of Ca²⁺ (A and C) or a large quantity of Ca²⁺ (B and D) and with GLA and green tea extracts (GTE) alone and in combination. The hatched column corresponds to the positive control. The black columns correspond to treatment of the keratinocytes with GLA alone, at different concentrations (1, 3 and 10 mg/ml). The white columns correspond to treatment of the keratinocytes with GTE alone, at different concentrations (1, 3 and 10 mg/ml). The grey columns correspond to treatment of the keratinocytes with a mixture of GLA and GTE at different concentrations (in mg/ml) (under each grey column, the figure on the left indicates the concentration of GTE and the figure on the right indicates the concentration of GLA). The x-axis corresponds to the concentrations of GLA, of GTE and of GLA/GTE mixture in mg/ml. The y-axis corresponds to the percentage expression relative to the control with Ca²⁺ at low (A and C) or at high (B and D) concentration.

### EXAMPLES

### Example 1

### Manufacture of a Dairy Product Containing a Combination of Gamma-Linolenic Acid and Green Tea Polyphenols

The dairy product is made by a conventional method of preparation of a stirred yoghurt.

In a first stage, skimmed milk, previously enriched in proteins by adding powdered milk or concentrated milk is preheated (95° C., 4 to 8 minutes) to remove bacterial contaminants. Then a stage of in-line incorporation of borage oil takes place prior to homogenization (50-300 bar, 40 to 95° C.). Heat treatment, maintaining close to pasteurization temperature and cooling are carried out successively. Then ferments are added and vat fermentation (30 to 45° C., 5 to 10 hours) takes place. Cutting of coagulum is then carried out, followed by cooling.

Green tea extract, vitamin E and optionally a fruit preparation are added in this stage. The mixture obtained is then packaged and stored in a cold room.

### Example 2

### Bioavailability Study

If GLA or catechins are absorbed by the oral route, their effects can only be exerted on the skin via the blood, which is the only vehicle capable of carrying these ingredients to all of the tissues in question (in the present case, the skin).

This study evaluates the bioavailability of the combination of catechins with GLA, and in particular the bioavailability of this combination in a yoghurt containing probiotics (see Table 1).

To evaluate this bioavailability, the plasma concentrations of GLA and of catechins are monitored at different points in time after ingestion of products 1, 2, 3 or 4.

According to data in the literature, a dose of approximately 300 mg/day of GLA produces an effect. In order to evaluate the absorption of GLA and its corresponding serum concentration, two doses of GLA were used: 300 and 150 mg/day.

Products Tested

Three types of products were developed and tested, as described in Table 1.

TABLE 1
Composition of the products tested in the bioavailability study

| | Fresh dairy product + | | Borage | Green tea |
|---|---|---|---|---|
| | active ingredients | | oil alone | extract alone |
| | Product 1 | Product 2 | Product 3 | Product 4 |
| Matrix | Dairy product fermented | | Ingredients | Ingredients |
| | with S. thermophilus, | | alone: | alone: green |
| | L. bulgaricus, L. casei* | | borage oil | tea extract in |
| | | | | an aqueous |
| | | | | solution |
| Borage | 1.5 g (equiva- | 0.75 g (equiva- | 1.5 g (equiva- | |
| oil | lent to 300 mg | lent to 150 mg | lent to 300 mg | |
| | of GLA) | of GLA) | of GLA) | |
| Green | 50 mg (equiva- | 50 mg (equiva- | | 50 mg |
| tea | lent to 45 mg | lent to 45 mg | | (equivalent |
| extract | of catechins) | of catechins) | | to 45 mg |
| | | | | of catechins) |

*Products 1 and 2 were made according to the method of manufacture described in Example 1.

Study Protocol

This study is a norm-centred, randomized, open study. It was carried out with 12 female volunteers (average age 30.8 years; body mass index (BMI) on average 21.4).

For each subject, the study took between 2 and 3 weeks.

Each subject made four 24-hour visits to the clinic: the first was the inclusion visit (V1) (1 week before the first assessment visit) and the other three were the assessment visits on D0 (V2), on D+4 (V3) and D+8 (V4).

At each visit, a clinical examination was carried out and a blood sample was taken for measuring the blood concentration of GLA and of catechins and for monitoring the kinetics of absorption.

After the first inclusion visit (V1), during the first week the subjects put into practice the dietary recommendations, which increased their awareness for reducing their consumption of food products containing large quantities of the ingredients tested (to limit any interference with the dosages applied). The three assessments were separated by at least 4 days and at most 7 days.

Protocol for the Assessment Visits

During each assessment visit (V2, V3, V4), the subjects consumed a product at random (among the 4 products). Thus, at the end of the third assessment visit, they had consumed each of the three test products.

Seven blood samples were taken in the 6 hours following ingestion of the product. The plasma concentration of the active ingredients was measured at 6 points in time (T0, T1 h, T2 h, T3 h, T4 h, T6 h) for measuring the plasma kinetics of GLA and the catechins.

Two additional samples were taken, so that differences in terms of absorption of the active ingredients could be identified more clearly:
T0.5 h was used for determining the bioavailability of the catechins in the plasma.
T5 h was used for determining the bioavailability of GLA in the plasma.

Plasma Analyses

The plasma concentrations of catechins and specifically of epicatechin (EC), epigallocatechin (EGC), and epigallocatechin gallate (EGCG) were determined by HPLC as described by Lee et al. (Lee M J, Prabhu S, Meng X, Li C, Yang C S. An improved method for the determination of green and black tea polyphenols in biomatrices by high-performance liquid chromatography with coulometric array detection. Anal. Biochem. 2000; 279: 164-9) and on the basis of the general knowledge of a person skilled in the art. The results are expressed in μmol/mL.

The GLA concentrations were found for the fraction of the chylomicrons obtained by isolation by ultracentrifugation. Extraction of the lipids of the chylomicrons was carried out according to the protocol of Moilanen et al. (Moilanen T, Nikkari T. The effect of storage on the fatty acid composition of human serum. Clin Chim Acta. 1981; 114:111-6). The measurements were carried out using gas chromatography. GLA was identified by methods known to a person skilled in the art, in particular by gas chromatography. The results are expressed in μg/mL.

Statistical Analyses

To evaluate the bioavailability of GLA and of the catechins, the following parameters were analysed:
the area under the curve (AUC) which gives information on the kinetics of bioavailability (g/mL/h),
the maximum concentration during the kinetic analysis (Cmax)(μg/mL),
the time during which the concentration is maximum (Tmax) (h).

A Student t test was used. The analysis was carried out on the population per protocol (PP) (n=11) because one of the 12 subjects included did not complete all the visits.

Results

Results for the Bioavailability of GLA

The bioavailability of GLA incorporated in a yoghurt (Product 1 or 2) or as a single ingredient administered via oil (Product 3), was measured in two ways:
AUC, Cmax and Tmax
the kinetics of GLA over the 6 hours following consumption for 11 subjects.

The results of the AUC, Cmax and Tmax analysis of GLA are shown in Table 2.

TABLE 2
GLA AUC, Cₘₐₓ and Tₘₐₓ analyses for each product.

| | AUC₀₋₆ₕ | Cₘₐₓ | Tₘₐₓ |
|---|---|---|---|
| | (μg/mL/h) | (μg/mL) | (h) |
| Product 1 | | | |
| (GLA 300 mg + Catechins | 27.9 ± 9.1* | 12.1 ± 7.03** | 2.00 ± 1.00*** |
| 45 mg in a fermented | | | |
| dairy product) | | | |
| Product 2 | | | |
| (GLA 150 mg + Catechins | 12.3 ± 3.1 | 6.5 ± 2.7 | 1.91 ± 1.58*** |
| 45 mg in a fermented | | | |
| dairy product) | | | |
| Product 3: Borage oil alone | 15.2 ± 10.5 | 9.4 ± 5.2 | 4.55 ± 1.29 |
| (equivalent to 300 mg GLA) | | | |

Results: mean value ± SD (standard deviation)
*statistically different from products 2 and 3 (p < 0.001).
**statistcally different from product 2 (p < 0.01).
***statistically different from product 3 (p < 0.01).

These results show an increase in the bioavailability of GLA absorbed via a fermented dairy product made according to the method implemented by the inventors.

It can also be noted that there is a response of the “dose-effect” type: the quantity of GLA absorbed was approximately twice as high in the case when 300 mg of GLA was consumed, relative to the case where 150 mg was consumed.

It can be deduced from these results that for ingestion of between 150 and 300 mg of GLA, a concentration of approximately 6 to approximately 12 μg/mL is found for the chylomicrons, which is equivalent to a concentration of between approximately 3 and approximately 6 μg/ml in the serum.

Using ultracentrifugation of the serum, the pellet consisting of the chylomicrons and the supernatant comprising the rest of the serum were separated. The proportion of the pellet relative to the rest of the serum is 50/50. Therefore a factor of ½ can be applied between the concentration of GLA in the chylomicrons and the concentration of GLA in the serum.

Results for the Bioavailability of the Catechins

The plasma concentrations of the three main catechins were measured at different points in time. It will be recalled that these catechins were as follows: epicatechin (EC), epigallocatechin (EGC) and epigallocatechin gallate (EGCG).

Statistical analysis showed significant differences in plasma concentrations of EGC at T1 h between products 1 and 2 and product 4. For the other two catechins, no significant difference was found. This shows that the catechins are as bioavailable in a milk matrix as alone in water.

It can be deduced from these results that for ingestion of 45 mg of catechins (EC, EGC, EGCG), a concentration of approximately 0.5 to approximately 2 μmol/L is found in the serum (EGCG: 0.04-0.33 μmol or 0.018-0.152 μg/ml, EGC: 0.05-0.14 μmol/l or 0.022-0.061 μg/ml, EC: 0.02-0.05 μmol/l or 0.005-0.014 μg/ml).

The inventors were able to relate these results to data in the literature (see in particular Navarro-Peran E. et al.: “The antifolate activity of tea catechins”, Enzymology Study Group, Department of Biochemistry and Molecular Biology A, Biology Faculty, Universidad de Murcia, Spain). In this publication, it is stated that between 0.1 and 1 μmol/l of EGCG (or approximately 0.03 to approximately 0.5 μg/ml of GTP) is found in the serum and tissues of tea drinkers. The consumption of GTP via the yoghurt described above is therefore virtually equivalent to that of someone who drinks tea every day.

### Example 3

### Clinical Study

The process of maturation (or process of epidermal differentiation) is very controlled and the balance of epidermal proliferation, differentiation and desquamation are key elements for skin function. The skin protects the body from excessive loss of water, this loss being expressed as TransEpidermal Water Loss (TEWL).

TEWL can be regarded as an indicator of the health of the skin. Its value depends on the place where it is measured, and depends on the season (it is higher during the winter months). However, for a given individual and for a given season, this value can very well be correlated to the health of the skin of this individual.

The skin can be mistreated owing to unfavourable environmental conditions or personal habits (cleaning, etc.), which can lead to dryness and to an increase in its sensitivity.

This study assesses how the “barrier” function of the skin is affected by the consumption of two portions per day for 6 months of a product containing a combination of GLA and of catechins relative to a control product (acidified milk containing neither probiotic, nor GLA, nor catechins).

Water loss is caused by evaporation, which can be determined by measuring the pressure gradient of the layer of water vapour above the skin. The technique of measurement of TEWL makes it possible to evaluate the “barrier” effect of the stratum corneum and of the hydrolipid film.

Methodology

This study is a norm-centred, randomized, double-blind, parallel study, carried out on female volunteers in good health, with dry, sensitive skin. The subjects (72) were divided into two groups of 36 subjects with an average age of 29.4±7.9 years and a body mass index (BMI) averaging 22.43±2.8.

One group received the test product whereas the other received the control product (without probiotic, without borage oil and without catechins) so as to allow comparison of the effects of these ingredients on the functionality of the skin.

The product compositions are summarized in Table 3.

TABLE 3
Composition of the products in the effectiveness study

| Quantity per portion | Test product | Control product |
|---|---|---|
| Borage oil (mg) | 750 | — |
| of which GLA (mg) | 150 | |
| Total green tea extract (mg) | 55 | — |
| of which GTP (mg) | 54 | — |
| of which catechins (mg) | 44 | — |
| L. casei DN-114 001 (CFU/g) | >5.10⁷ | — |
| Lactobacillus bulgaricus and Streptococcus | >10⁷ | — |
| thermophilus (CFU/g) | | |
| Vitamin E (mg) | 2 | 0 |

The study lasted 7 months. It was divided into two parts: a selection phase (4 weeks) and a consumption phase (24 weeks). The subjects underwent 4 assessment visits at 6-week intervals during the period of consumption of the product. A period of consumption of 6 months makes it possible to compare the effect of the product over an extended period of time and this also makes it possible to compare the effect in relation to the seasons.

The first objective of this study was to determine whether the consumption of a fermented dairy product for 12 weeks could improve the “barrier” function of the skin of the subject's forearm. This was evaluated by measuring the TEWL with sodium lauryl sulphate (SLS). The TEWL was measured on the inner part of the forearm with an EVAPORIMETER EP2® and expressed in g/m²/h. (SERVOMED, Sweden).

The second objective of this study was to determine whether the consumption of the dairy product containing the combination of the invention could improve the “barrier” function of the skin of the forearm over time, by measuring the TEWL in SLS.

Other parameters were measured, such as hydration, elasticity, blood markers of inflammation and the overall quality of the skin by clinical assessment and a personal questionnaire.

The study was started in autumn and continued until spring, so as to compare the effect of the product at different times of the year. It will be recalled that the “barrier” function of the skin is thought to deteriorate during the winter months. The analysis was carried out on all the subjects.

Statistical Analysis

The data were analysed in two ways:
comparison between two groups at different points in time (6, 12, 18 and 24 weeks) using a non-parametric ANOVA (analysis of variance) test (owing to the distribution, the values are expressed in medians rather than averages);
comparison of the evolution of the effect over time; this more general analysis of the response kinetics makes it possible to take account of the differences between the two products and environmental variations; an ANOVA test based on measurements repeated over two periods of time (total study of 24 weeks and up to 12 weeks (first criterion of the study)) was used.

These analyses take account of the reference levels in each case.

Results

Intention-to-Treat (ITT) population

Analysis of the ITT population (n=67) showed an improvement (supported by data) of the “barrier” function of the skin evaluated by the TEWL (without SLS), for subjects who consumed the test product relative to those who consumed the control product, throughout the study.

There is a natural variation in the “barrier” function (for example a slight deterioration in winter shows an increased value of the TEWL) but the active product tends to be superior to the control at 6 weeks and, at 8 weeks, the differences are statistically very significant. There are no significant differences at 12 and 24 weeks between the treatments with the test product and the control product (mean values).

Comparison of the relative percentages of the increase in the values of the TEWL in the two groups shows the largest difference (27.5%) at 18 weeks.

At 24 weeks, an improvement of the “barrier” function of the test group was observed (negative value of the TEWL compared with the reference level) suggesting an overall cumulative effect of consumption of the product.

Analysis of the effects over time reveals that the test product reduces the TEWL significantly during the study (24 weeks of consumption [p=0.02]) and also in the phase up to 12 weeks of consumption ([p=0.036]).

On average, over the whole study period, the difference in the TEWL between the two groups is 14%, indicating that the consumption of the product causes a 14% increase in water retention by the skin.

Subgroup BMI<25

For the subgroup with a body mass index (BMI)<25 (n=23), variations in the TEWL due to the change of season were observed. These results are similar to those observed in the ITT population.

The TEWL of the test product compared significantly with that of the control product at 6, 12 and 18 weeks. A positive trend was also observed after 24 weeks of consumption. Expressed in percentage TEWL, the results reveal a 15% and 25% increase in the “barrier” function of the test group relative to the control group after, respectively, 6 and 18 weeks of consumption.

Analysis of the effect over time shows significant differences during the period up to 24 weeks [p=0.0031] and also during the period up to 12 weeks [p=0.0039]. On average, over the whole study period, the difference in the TEWL between the two groups is 22.3%, indicating that consumption of the product causes a 22.3% increase in water retention by the skin.

Secondary Parameters

Among the secondary parameters (hydration, elasticity, inflammatory markers in the blood, etc.), statistical analysis reveals improvements in the overall quality of the skin as evaluated by a questionnaire for self-assessment of the firmness and health of the skin at 12 weeks.
firmness: 46.2% of the subjects in the test group felt that their skin was firmer; compared to 11.5% of the subjects in the control group;
health: 46.2% of the subjects in the test group described their skin as being healthier than the subjects in the control group (7.7%).

In order to see the direct effect of these doses of GTP and of GLA on the cells of the skin, the inventors tested ranges of concentrations of these active ingredients in vitro.

### Example 4

### In Vitro Study of the Induction of Keratinocyte Differentiation

The effect of GLA and of GTP, as well as of their combination, on keratinocyte differentiation, is quantified on the basis of protein markers of maturation:
transglutaminase K (TGK) (Lee Y. S. et al. “Differentiation of cultured human epidermal keratinocytes at high cell densities is mediated by endogenous activation of the protein kinase C signaling pathway”. J Invest Dermatol. 1998 November; 111(5):762-6; Eckert et al. R. L. Transglutaminase function in epidermis. J. Invest. Dermatol. 124:481-492, 2005), and
involucrin (Candi et al. “The Cornified envelope: a model of cell death in the skin”, Nature Publishing Group. April 2005, vol. 6).

Transglutaminase K is strongly expressed in the keratinocyte when it reaches an advanced phase of maturation (corresponding to the start of the transformation to corneocyte). Transglutaminase K is involved in the synthesis of the ceramides of the extracellular matrix of the stratum corneum, thus participating in the maintenance of good hydration of the skin.

Involucrin is a protein that is included in the composition of the stratum corneum. It is therefore a structural protein of the cornified envelope.

GLA and GTP, alone and in combination, are tested in a model in vitro in order to:
support their beneficial effect on the maturation of keratinocytes through the expression of TGK, and of involucrin, and
evaluate their potential synergistic effect through the expression of TGK and of involucrin.

1) Effect of Gamma-Linolenic Acid (GLA) and of Green Tea Polyphenols (GTP) as Well as their Combination on the Expression of Transglutaminase K

Nature of the Active Ingredients Tested:

The GLA used is the product L2378 from the company SIGMA. The 4 main forms of catechins forming green tea polyphenol are mixed in the natural proportions: EGCG, EGC, ECG and EC (respective SIGMA references E4143, E3768, E3893 and E4018).

The range of concentration of GLA tested in vitro was from 1 to 4 μg/ml and that of GTP was from 0.3 to 0.7 μg/ml.

Conditions for Culture of Nheks:

The tests are carried out on normal human keratinocytes (NHEKs) in a monolayer. The NHEKs are sown at 10⁴/well in serum-free medium (SFM) without calcium, under conditions of temperature of 37° C., CO₂ of 5% and relative humidity RH>95%. The NHEKs are incubated like this for 24 hours. The medium is then removed. Then the NHEKs are incubated for 48 hours in the presence of either:
SFM (serum-free medium) with addition of gamma-linolenic acid and green tea polyphenols at a concentration of 1.25, 2.5 or 3.75 μg/mL for GLA and at a concentration of 0.5 μg/ml for GTP;
SFM (serum-free medium) without calcium, which serves as negative control;
SFM (serum-free medium) with addition of calcium (1.50 mM), which serves as positive control.

The media are removed and a second incubation is carried out in the presence of the 3 types of media.

Cytotoxicity Test

Firstly, to evaluate the maximum concentration of the two active ingredients tolerated by the NHEKs under these conditions, a cytotoxicity test is undertaken.

The cell viability of the NHEKs is evaluated after labelling with 3-[4,5-dimethylthiazol-2-yl]-2,5-diphenyltetrazolium bromide (MTT) (SIGMA).

Thus, for GLA the maximum concentration without loss of viability of the NHEKs is 5.00 μg/mL.

For the GTP mixture, the maximum concentration without loss of viability of the NHEKs is 1.00 μg/mL.

Quantification of the Marker of Cell Differentiation

The culture medium is removed and then rinsing with PBS is carried out. The cells are then permeabilized and fixed with absolute methanol for 10 minutes at −20° C. Then drying and nonspecific saturation with a PBS-Tween20-milk proteins mixture are carried out, followed by rinsing with PBS. The NHEK cells are labelled with an anti-TGK monoclonal antibody (Reference: TEBU 0175003), then incubated for 1 hour at room temperature and rinsed with PBS.

Detection is carried out with a GAM-FITC conjugated secondary antibody (Reference: Life Technology A 11001) and the fluorescence is measured using software (Reference: NIKKON; LICIA Software).

The nuclei are labelled and counted by the Hoechst technique. First, the medium is removed and rinsing with PBS is carried out. Hoechst labelling (Reference: bis-benzimide Sigma B1155) is then used and incubation is carried out for 1 hour at ambient temperature. This is followed by rinsing with PBS, image acquisition and quantification of fluorescence.

The results are expressed in specific fluorescence of TGK, i.e. they are related to the number of nuclei so as to be independent of the variations in growth of the cells.

Moreover, all these results are also related to the results of the negative control (which represents normal differentiation), which is fixed at 100%.

Statistical Analysis of the Results:

Statistical analysis of the results is carried out using the software JUMP 6 SAS Institute.

A Dunnett test (specialized multiple comparison test, for example comparisons made only with the control group against all the other groups) makes it possible to confirm the significance of the results in comparison with the untreated control condition.

Results:

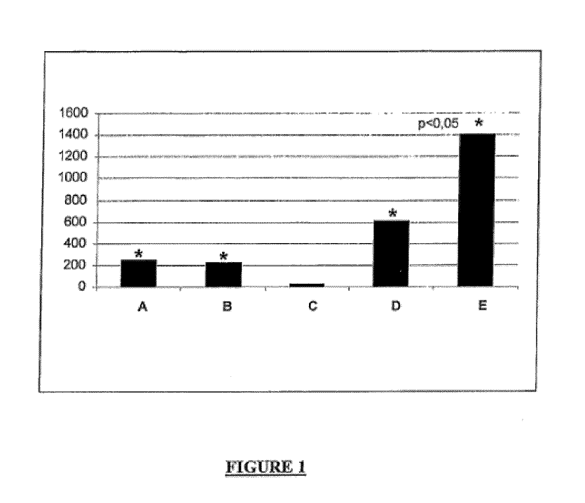

The results are presented in FIG. 1.

| | Expression of | |
|---|---|---|
| | TGK (average) | p_{dunnet} TGK |
| Untreated negative control | 0 | — |
| GLA 1.25 μg/mL | 250 | 0.012 |
| GLA 3.75 μg/mL | 220 | 0.026 |
| GTP 0.5 μg/mL | 29 | 0.98 |
| GLA 2.5 μg/mL + GTP 0.5 μg/mL | 605 | <0.0001 |
| Positive control Ca++ | 1407 | <0.0001 |

The average expression of TGK corresponds to measurement of the quantity of fluorescence referred to the number of nuclei after Hoechst labelling.

The term p_{dunnett} TGK corresponds to the probability of making an error by saying that a value is significantly different from the value of the negative control (if p<5%, this signifies that there is a significant difference, if 5<p<10% this signifies that there is a trend).

The values tested in vitro are values representative of the range of serum concentrations of GLA and GTP found.

The results are presented in FIG. 1.

Conclusions:

GLA Alone:

The expression of TGK after treatment with GLA seems to be activated.

This activation seems to be similar for the two concentrations tested.

This positive effect of GLA is in agreement with data in the literature.

GTP Alone:

Treatment with GTP does not appear to significantly increase the natural maturation in this model at the concentration tested.

(A result reported in the literature is the effect of activation of the promoter of involucrin by EGCG at the high dose of 20 μg/mL).

GLA and GTP Combination:

The activation of TGK by a treatment containing GLA and GTP is greater than that provided by GLA alone. There is therefore a synergistic effect, in that the addition of a concentration of GTP, a priori without observable effect under these conditions, greatly increases the activation of TGK by GLA alone.

These results are to be considered in conjunction with those of the clinical study (results for the TEWL).

2) Effect of Gamma-Linolenic Acid (GLA) and of Green Tea Extracts (GTE) as Well as their Combination on the Expression of Involucrin

Nature of the Active Ingredients Tested:

The GLA used is the product L2378 from the company SIGMA. The GLA was dissolved in DMSO and BSA without free fatty acids.

The green tea extract used is the Taiyo Chemicals product. It was dissolved in sterile water.

Conditions for Culture of the NHEKs:

The tests are carried out on normal human keratinocytes (NHEKs) in monolayer.

The keratinocytes are obtained from women subjects and come either from the abdominal epithelium (group 1) or from the epithelium of the breast (group 2).

The NHEKs of the two groups are incubated for 48 hours in the presence of a low (0.3 μg/ml) or a high (1.2 μg/ml) concentration of calcium, and in the presence of either:
SFM (serum-free medium) with addition of 0, 1, 3 and 10 μg/mL of gamma-linolenic acid and/or of green tea extracts, in which the concentration of green tea polyphenol is approximately 88 wt. %;
SFM (serum-free medium) with addition of calcium, which serves as positive control.

The dynamics of calcium in the keratinocytes is one of the factors determining their differentiation from the stem cells.

After culture for 48 hours, the cells are harvested and are then lysed. The proteins are recovered and are then assayed by the ELISA technique.

### Cytotoxicity Test

The cell viability of the NHEKs is evaluated after labelling with 3-[4,5-dimethylthiazol-2-yl]-2,5-diphenyltetrazolium bromide (MTT) (SIGMA).

The values tested in vitro are values representative of the range of GLA and GTP concentrations found in the serum.

The cytotoxicity results show that the GTE and GLA concentrations are not toxic to the NHEKs up to 10 μg/mL, regardless of the calcium concentration.

Quantification of Expression of Involucrin

The experiments described below were repeated 6 times.

The expression of involucrin was determined by an ELISA test.

The range of GLA concentration tested in vitro is from 0 to 10 μg/ml and that of GTE is also from 0 to 10 μg/ml.

This range is selected in relation to the validation curve (absorbance as a function of concentration) previously developed with an internal standard (assayed involucrin) supplied in the kit.

The absorbances of the cell lysates obtained previously are to be linked to the concentrations of involucrin, in relation to the standard curve.

Results:

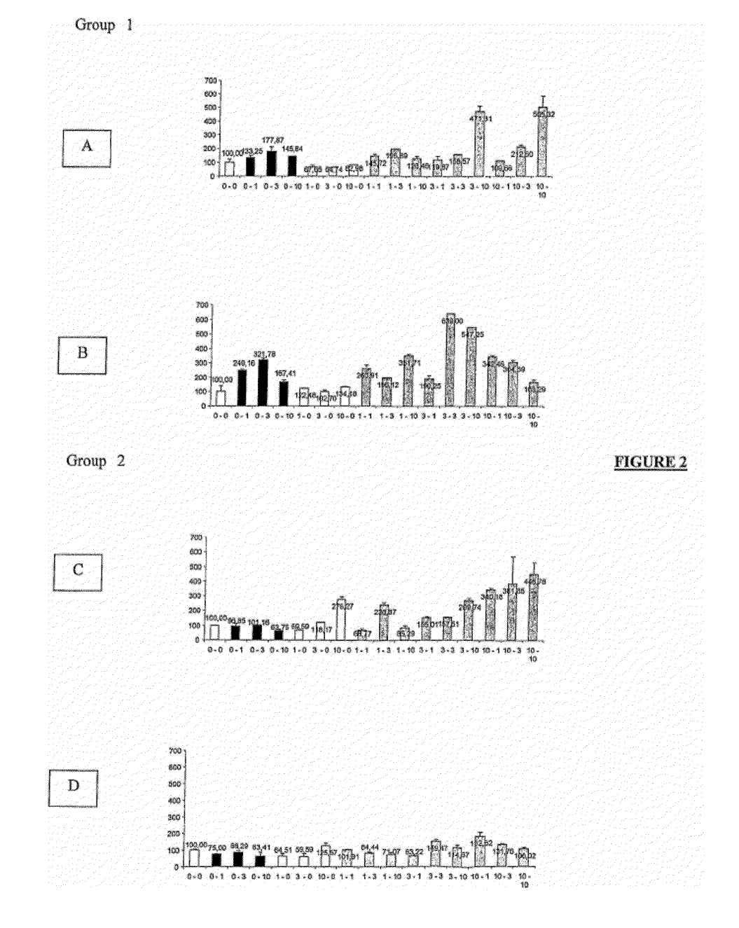

The results are presented in FIG. 2.

GLA Alone:

Expression of involucrin is increased after treatment of the NHEKs of groups 1 and 2 with GLA alone (0 to 10 μg/mL) under conditions of high or low calcium concentration, as defined above.

GTE Alone:

Expression of involucrin is increased after treatment of the NHEKs of groups 1 and 2 with GTE alone (0 to 10 μg/mL) under conditions of high or low calcium concentration, as defined above.

GLA and GTE Combination:

The combination of GLA and GTE has a synergistic effect on the expression of involucrin.

In fact activation of the expression of involucrin by the treatment containing GLA and GTE is greater than that obtained with GLA alone or GTE alone.

This synergistic effect can be observed both in group 1 and in group 2, attesting to the relevance of the results observed.

1-27. (canceled)

28. Method for increasing keratinocyte differentiation in vitro and ex vivo by means of a combination comprising gamma-linolenic acid and at least one green tea polyphenol, wherein the ratio of gamma-linolenic acid to green tea polyphenol varies from 1 to 10, and in particular varies from 2 to 8 and still more particularly from 4 to 6.

29. The method according to claim 28, wherein the green tea polyphenol is selected from compounds of the flavan-3-ol class of flavonoids.

30. The method according to claim 28, wherein the green tea polyphenol is selected from epigallocatechin gallate (EGCg), epigallocatechin (EGC), epicatechin (EC), gallocatechin (GC), gallocatechin gallate (GCg), epicatechin gallate (ECg) and catechin (C).

31. The method according to claim 28, wherein the green tea polyphenol is contained in a plant extract including green tea extract.

32. The method according to claim 28, wherein the gamma-linolenic acid is contained in a vegetable oil including borage oil, primrose oil or blackcurrant seed oil.

33. Combination comprising gamma-linolenic acid and at least one green tea polyphenol, wherein the ratio of gamma-linolenic acid to green tea polyphenol varies from 1 to 10, and in particular varies from 2 to 8 and still more particularly from 4 to 6.

34. Combination comprising gamma-linolenic acid and at least one green tea polyphenol, wherein the ratio of gamma-linolenic acid to green tea polyphenol varies from 1 to 10, and in particular varies from 2 to 8 and still more particularly from 4 to 6, and in which the total quantity of gamma-linolenic acid and of polyphenol is 100 to 500 mg, in particular 150 to 400 mg, more particularly 350 mg.

35. The composition comprising a combination according to claim 33, wherein,
the concentration of gamma-linolenic acid is 0.1 to 0.5%, more particularly 0.1 to 0.3%, still more particularly 0.15 to 0.3 wt. % relative to the total weight of the composition and
the concentration of green tea polyphenol is 0.01 to 0.1%, more particularly 0.02 to 0.07%, still more particularly 0.045 wt. % relative to the weight of the composition.

36. The composition according to claim 35, wherein the green tea polyphenol is selected from compounds of the flavan-3-ol class of flavonoids.

37. The composition according to claim 35, wherein the green tea polyphenol is selected from epigallocatechin gallate (EGCg), epigallocatechin (EGC), epicatechin (EC), gallocatechin (GC), gallocatechin gallate (GCg), epicatechin gallate (ECg) and catechin (C).

38. The composition according to claim 35, wherein the green tea polyphenol is contained in a plant extract including green tea extract.

39. The composition according to claim 35, wherein the gamma-linolenic acid is contained in a vegetable oil including borage oil, primrose oil or blackcurrant seed oil.

40. Composition according to claim 35, wherein the concentration of gamma-linolenic acid is 0.15 to 0.3 wt. % relative to the total weight of the composition.

41. Composition according to claim 35, wherein the concentration of green tea polyphenol is 0.045 wt. % relative to the total weight of the composition.

42. Composition according to claim 35, wherein the concentration of gamma-linolenic acid is 0.15 to 0.3 wt. % relative to the total weight of the composition and the concentration of green tea polyphenol is 0.045 wt. % relative to the total weight of the composition.

43. Oral composition comprising a combination according to claim 33, being in the form of a food product, a food supplement, or a cosmetic composition.

44. The oral composition according to claim 43, characterized in that the food product is selected from the group comprising a fresh dairy product, a yoghurt, a fromage frais, a fermented dairy product, a dessert, a drink, a liquid, a cream, a fruit and/or vegetable purée.

45. The oral composition according to claim 43, characterized in that the food supplement is selected from the group comprising coated tablets, pills, gelatin capsules, syrup, gel, powder for reconstitution, paste, gum.

46. The oral composition according to claim 43, characterized in that the cosmetic composition is selected from the group comprising creams, emulsions, lotions.

47. The oral composition according to claim 43, comprising a combination in which the concentration of gamma-linolenic acid is 0.15 to 0.3 wt. % relative to the total weight of the composition and the concentration of green tea polyphenol is 0.045 wt. % relative to the total weight of the composition.

48. Cosmetic product comprising a combination according to claim 33, in combination with a suitable excipient for administration by the topical route.

49. The cosmetic product according to claim 48, wherein the combination in which the concentration of gamma-linolenic acid is 0.15 to 0.3 wt. % relative to the total weight of the composition and the concentration of green tea polyphenol is 0.045 wt. % relative to the total weight of the composition.

50. Pharmaceutical composition containing, as active ingredient, a combination according to claim 33, in combination with a pharmaceutically acceptable vehicle.

51. Method of cosmetic treatment, comprising the absorption by the oral route of a cosmetic composition according to claim 46.

52. Method of cosmetic treatment, comprising the topical application of a cosmetic product according to claim 48.

53. Method for improving the quality of the skin by acting on the “barrier” function of the skin, comprising the administration of a combination according to claim 33 to a patient in need thereof.

54. Method for the prevention or treatment of pathologies involving a change, in particular a decrease, in keratinocyte differentiation, comprising the administration of a combination according to claim 33 to a patient in need thereof.
